(19) United States
(12) Patent Application Publication
(10) Pub. No.: US 2003/0049668 A1
(43) Pub. Date: Mar. 13, 2003

(54) Method for determining quinolon resistance of tubercle bacilli

(76) Inventors: Yasuhiko Suzuki, Osaka-shi (JP); Tatsuo Ichihara, Chiba-shi (JP); Noriko Satoh, Chiba-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/219,219

(22) Filed: Aug. 15, 2002

(30) Foreign Application Priority Data
Sep. 4, 2001 (JP) 2001-267769

(51) Int. Cl. C12Q 1/68; C12N 1/20

(52) U.S. Cl. 435/6; 435/252.3

(57) ABSTRACT

A novel mutation in the gyrase A gene involved in quinolon resistance phenotype acquisition of tubercle bacillus is identified and utilized to provide a method and kit for determining quinolon resistance of tubercle bacillus.

### BACKGROUND OF THE INVENTION

1. Field of the Invention

The present invention relates to a method and kit for determining quinolon resistance of srtains of tubercle bacilli (Mycobacterium tuberculosis), more specifically, a method and kit for determining quinolon resistance by detecting a novel mutation in the gyrase A gene, which imparts the quinolon resistance phenotype to tubercle bacilli.

2. Description of the Related Art

Drug resistance phenotype acquisition mechanisms of bacteria include (1) such change of the cell wall structure that permeation of a drug into a cell should be blocked, (2) acquisition of a gene coding for an enzyme that decomposes or inactivates a drug, (3) change of a compound in a cell as a target of drug and so forth. Acquisition of these phenotypes are acheived by transmission among bacteria via transposable genetic elements such as plasmid and transposon, change of chromosomes or the like.

Acquisition of resistance phenotype to drugs (hereinafter, referred to as “antituberculous drug”) by Mycobacterium tuberculosis (hereinafter, also referred to as “tubercle bacilli”) is attributable to a spontaneous chromosomal mutation (A. Telenti, Tuberculosis, 1997, 18 (1): 55-64). For example, the mechanism of resistance of tubercle bacilli to rifampicin, which is an antituberculous drug, is resulted from nucleotide substitution occurring in the gene encoding the RNA polymerase β subunit protein, which is a target substance of rifampicin (A Telenti et al., Lancet, 1993, 341: 647-650). Furthermore, nucleotide substitution in the rpsl and rrs genes or the like induces streptomycin resistance (Y. Suzuki, et al., J. Applied. Microbiol., 1997, 83: 634-640). Thus, mechanisms for acquisition of resistance phenotype by a mutation in a gene involved in each target molecule exist for some antituberculous drugs.

Accordingly, most of drug resistance phenotype acquisition mechanisms of tubercle bacilli are explained based on spontaneous mutations.

In Escherichia coli, bacteria belonging to the genus Staphylococcus and so forth, quinolon resistance is acquired by introduction of a nucleotide mutation into a gene encoding the DNA gyrase activity, topoisomerase IV activity or a membrane integral protein involved in uptake and excretion of a substance into and from a cell (Cambau E., et al., Res. Microbiol., 1996, 147: 52-59; Ferrero L., et al., Antimicrob. Agents Chemother., 1995, 39: 1554-1558) or the like. One of the target compounds of quinolon is gyrase A, and a mutation in the gyrase A gene greatly contributes to resistance to clinically important quinolon molecular species (ciprofloxacin, ofloxacin etc., Cambau E., et al., J. Infect. Dis., 1994, 170: 479-483).

There have also been reported several mutations in the gyrase A gene involved in acquisition of quinolon resistance phenotype in tubercle bacilli (Antimicrob. Agents Chemother., 1996, 40 (8), 1768-1774; J. Infect. Dis., 1996, 174, 1127-1130; Eur. J. Clin. Microbiol. Infect. Dis., 1997, 16, 395-398; J. Infect. Dis., 2000, 182, 517-525).

### SUMMARY OF THE INVENTION

An object of the present invention is to provide a method and kit for determining quinolon resistance of tubercle bacilli by identifying a novel mutation in the gyrase A gene involved in acquisition of quinolon resistance phenotype in tubercle bacilli and utilizing the mutation.

The inventors of the present invention assiduously studied in order to achieve the aforementioned object. As a result, they found a novel mutation imparting a quinolon resistance phenotype in the gyrase A gene sequence of tubercle bacilli and accomplished the present invention.

That is, the present invention provides the followings.

(1) A method for determining quinolon resistance of a Mycobacterium tuberculosis strain by detecting a mutation in a gyrase A gene of the bacterial strain, wherein the mutation is substitution of another amino acid residue for an amino acid residue corresponding to the 89th aspartic acid residue in an amino acid sequence encoded by the gyrase A gene.

(2) The method according to (1), wherein the other amino acid residue is an asparagine residue.

(3) The method according to (1) or (2), wherein the amino acid sequence encoded by the gyrase A gene is the amino acid sequence of SEQ ID NO: 4 or an amino acid sequence of SEQ ID NO: 4 including substitution, deletion or insertion of one or several amino acid residues.

(4) The method according to any one of (1) to (3), wherein the mutation is substitution of T for G that is the first nucleotide of a codon coding for an aspartic acid residue.

(5) The method according to (4), wherein the mutation occurs at a position corresponding to the 530th nucleotide in the nucleotide sequence of SEQ ID NO: 3.

(6) The method according to any one of (1) to (5), further comprising detecting a substitution of A for a base corresponding to G at the 545th position in the nucleotide sequence of SEQ ID NO: 3.

(7) A kit for determining quinolon resistance of a Mycobacterium tuberculosis strain by detecting a mutation in a gyrase A gene of the bacterial strain, comprising an oligonucleotide for detecting a substitution of another amino acid residue for an amino acid residue corresponding to the 89th aspartic acid residue in an amino acid sequence encoded by the gyrase A gene.

A novel mutation in the gyrase A gene involved in quinolon resistance phenotype acquisition by tubercle bacilli was identified by the present invention. By utilizing this mutation, a novel method and kit for determining quinolon resistance of tubercle bacilli are provided.

### BRIEF EXPLANATION OF THE DRAWINGS

FIG. 1 shows alignment of nucleotide sequences of gyrA genes from strains of tubercle bacilli resistant to quinolon.

### DETAILED DESCRIPTION OF THE PREFERRED EMBODIMENTS

Hereafter, the present invention will be explained in detail.

The present invention provides a method for determining quinolon resistance of a tubercle bacillus strain by detecting a mutation in the gyrase A gene (hereinafter, referred to as “gyrA”) of the bacterial strain. Within the reported nucleotide sequence of the gyrA gene of wild tubercle bacillus strain (GenBank locus MSGGYRAB, accession L27512.1 GI: 1107467), the sequence of nucleotide numbers 2041-4923 is shown as SEQ ID NO: 3. Further, an amino acid sequence encoded by the nucleotide sequence is shown as SEQ ID NO: 4. The amino acid sequence is shown in both of SEQ ID NOS: 3 and 4.

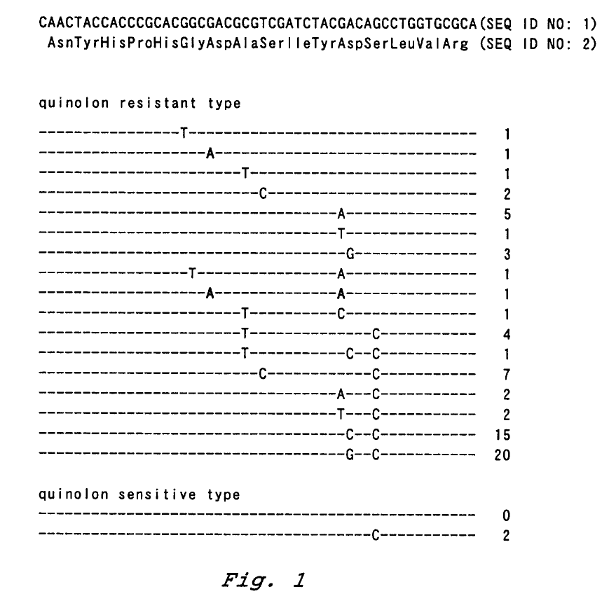

The method of the present invention is characterized in that the mutation in the aforementioned gyrA gene is a substitution of another amino acid residue for an amino acid residue corresponding to the 89th aspartic acid residue in the amino acid sequence encoded by the gyrA gene. FIG. 1 shows comparison of nucleotide sequences of a region around the mutation point. Further, a nucleotide sequence of the aforementioned region in the gyrA gene of a wild strain is shown as SEQ ID NO: 1. The amino acid sequence encoded by this sequence is shown as SEQ ID NO: 2. SEQ ID NO: 1 corresponds to the nucleotide numbers 511-560 in the nucleotide sequence including the gyrA gene shown as SEQ ID NO: 3. Further, the amino acid sequence of SEQ ID NO: 2 corresponds to the amino acid numbers 83-98 in the amino acid sequence encoded by the gyrA gene and shown as SEQ ID NO: 4.

As the aforementioned other amino acid residue, an asparagine residue can be mentioned. As a mutation in the gyrA gene that induces a mutation for substituting an asparagine residue for the aspartic acid residue, substitution of T for G that is the first nucleotide of a codon coding for an aspartic acid residue can be mentioned. A specific example of this mutation is a mutation occurring at a position corresponding to the 530th nucleotide in the nucleotide sequence of the gyrA gene shown as SEQ ID NO: 3.

The nucleotide or amino acid numbers in the nucleotide sequence or the amino acid sequence shown as SEQ ID NO: 3 or 4 may be changed due to a mutation in the gyrA gene occurring at a position that is not within a codon corresponding to the 89th aspartic acid residue. A gyrase A having such a mutation has an amino acid sequence including substitution, deletion or insertion of one or several amino acids in the amino acid sequence shown as SEQ ID NO: 4. The present invention can also be applied to a gene coding for the gyrase A including such a mutation so long as the mutation is involved in quinolon resistance. The expressions “amino acid residue corresponding to the 89th aspartic acid residue” and “position corresponding to the 530th nucleotide” refer to an amino acid residue or a nucleotide residue corresponding to such a position in the sequence shown as SEQ ID NO: 3 or 4 when the nucleotide numbers or amino acid numbers are changed as described above. For example, when the first amino acid residue of SEQ ID NO: 4 is deleted, an amino acid residue corresponding to the 89th aspartic acid residue means an aspartic acid residue that is the 88th amino acid residue from the N-terminus.

In the present invention, known mutations in the gyrA gene involved in quinolon resistance and/or mutations yet to be found in future may be detected in addition to the substitution of an amino acid residue corresponding to the 89th aspartic acid residue. As such mutations, there can be mentioned substitution of A for a base corresponding to G at the 545th position in SEQ ID NO: 3.

Mutations in the gyrA gene of tubercle bacillus can be detected in the same manner as in usual mutation detecting methods so long as a single nucleotide polymorphism can be detected. For example, there can be mentioned a method for detecting a mutation by directly determining the nucleotide sequence of the gyrA gene. Further, examples of a method for detecting a mutation utilizing PCR include methods such as the template-directed dye-terminator incorporation method (Chen, X. et al., Proc. Natl. Acad. Sci. USA, 20, 10756-10761 (1997)), the dye-labeled oligonucleotide ligation method (Chen, X. et al., Genome Res., 5, 549-556 (1998)), the molecular beacon method (Tyagi, S. et al., Nat. Biotechnol., 1, 49-53 (1998)), the dynamic allele-specific hybridization method (Howellm, W. M. et al., Nat. Biotechnol., 1, 87-88 (1998)) and so forth.

Further, as methods that do not utilize PCR, there can be mentioned the padlock probe method developed by Mats Nilsson et al. in 1994 (Nilsson, M. et al., Science, 5181, 2085-2088 (1994)) and modified by Paul M. Lizardi et al. (Lizardi, P. et al., Nat. Genet., 3, 225-232 (1998)), the invader assay method (WO94/29482) developed by Victor Lyamichev and so forth.

Furthermore, as other methods for detecting a single nucleotide polymorphism, the Luminex method utilizing flow cytometry and methods utilizing matrix-assisted laser desorption ionization mass spectrometry (MALDI-TOFMS) are also known and can be used for the present invention.

Further, microarray methods (WO95/11995, U.S. Pat. No. 5,837,832), which are widely used in recent years, such as the single base sequencing on oligomicroarray method (Drmanac, R. et al., Genomics, 2, 114-128 (1989)) can also be used. The microarray methods are techniques in which a large number of capture nucleic acids are immobilized on a solid phase, a labeled target nucleic acid is hybridized with the capture nucleic acids to be captured, and the presence or absence or existence ratio of the target nucleic acid is analyzed exhaustively.

As an example of methods preferably used in the present invention, a method of detecting a target mutation in the gyrA gene by using an oligonucleotide immobilized on a substrate will be shown below.

As the oligonucleotide immobilized on the substrate, used is an oligonucleotide having a nucleotide sequence of a region including the position of the aforementioned mutation in the gyrA gene (hereinafter, referred to as “capture oligo”).

When the capture oligo is designed, it is usually preferred that a position corresponding to the aforementioned mutation included in the capture oligo should exist in the center portion of the capture oligo. When the capture oligo is too short, detection of the hybridization becomes difficult. When it is too long, inhibition of the hybridization due to a sequence unique to that type does not occur. Therefore, a length in the range of 10 to 24 nucleotides is preferred. Further, when there is a secondary structural failure that negatively affects the hybridization in a process of hybrid formation of the capture oligo and a target described later, a spacer or a nucleotide that does not form a hydrogen bond with any nucleotide can be introduced into the oligonucleotide sequence to avoid the aforementioned failure.

Although DNA is usually used as the capture oligo, a peptide nucleic acid (PNA) may also be used. Since a hybrid formed by a peptide nucleic acid with a nucleic acid derived from a test human genome has a higher Tm (melting temperature) in comparison with that obtained by using an oligonucleotide, it is expected that a stable hybridization signal can be obtained. The peptide nucleic acid can be readily synthesized by using a usual peptide synthesizer.

Synthesis of the oligonucleotide, preparation of chromosomal DNA, hybridization and PCR can be performed according to usual methods well known to those skilled in the art (refer to Maniatis, T. et al., “Molecular Cloning A Laboratory Manual, Second Edition”, Cold Spring Harbor Laboratory Press (1989)). Further, the oligonucleotide can be synthesized by using a commercially available DNA synthesizer.

Material of the substrate on which the oligonucleotide is immobilized is not particularly limited so long as the oligonucleotide can be stably immobilized thereon. However, for example, glass, synthetic resins such as polycarbonate and plastic can be mentioned. Although shape of the substrate is not also particularly limited, a plate-like or film-like substrate can be mentioned. The substrate preferably has a uniform and planar surface.

The oligonucleotide can be immobilized on the substrate by using a method used in a usual hybridization method such as physical adsorption, electrical coupling and formation of a molecular covalent bond. For example, there can be mentioned a method using a substrate of which surface is coated with a compound having a carbodiimide group or an isocyanate group (Japanese Patent Laid-open Publication (Kokai) No. 08-023975). The substrate coated with a compound having a carbodiimide group or an isocyanate group on the surface thereof can be prepared by coating the substrate surface with a macromolecular compound having a carbodiimide group or an isocyanate group. The oligonucleotides can be immobilized with a covalent bond on the substrate by irradiating with a ultraviolet ray. Further, as a linker for bonding the carbodiimide group or isocyanate group and the oligonucleotide, a compound having an amino group or an imino group having high reactivity with the carbodiimide group or isocyanate group is used. In the case of imino group, the compound can be bonded with a carbodiimide group or an isocyanate group by polymerizing thymine to either one of the ends of capture oligo.

The oligonucleotide can be immobilized on the substrate by, for example, spotting an oligonucleotide solution on the substrate by using a spotting machine. Usually, the oligonucleotide solution is preferably spotted in a substantially circular shape.

Further, multiple kinds of oligonucleotide are each usually spotted at multiple positions on a single substrate, and these spots are preferably arranged in a grid pattern. When the spot size is 1000 μm in diameter, the total number of spots is preferably 1600 spots/cm² or less and 40×40 or less of spots are preferably provided when they are spotted in a square pattern. Further, when the spot size is 10 μm in diameter, the total number is preferably 400 spots/cm² or less, and 20×20 or less of spots are preferably provided when they are spotted in a square pattern. Further, when the vertical and horizontal sizes of the pattern are different, the numbers of the spots along the vertical and horizontal directions may be adjusted depending on shape of the pattern.

The presence or absence of a specific mutation in the gyrA gene can be determined by hybridizing a nucleic acid fragment probe including a region corresponding to the position of the aforementioned mutation in the gyrA gene of chromosomal DNA of tubercle bacillus (referred to as “nucleic acid probe”) with a wild type capture oligo and a mutant capture oligo and examining with which type of capture oligo the probe hybridized. That is, to which drug the strain has resistance can be detected depending on the hybridization position on the substrate.

The nucleic acid can be prepared from tubercle bacilli in the same manner as in a usual method for preparing nucleic acids from bacteria. For example, DNA can be prepared according to the method described in Maniatis, T. et al., “Molecular Cloning A Laboratory Manual, Second Editions”, Cold Spring Harbor Laboratory Press (1989).

The nucleic acid probe can be prepared by amplifying a nucleic acid by using primers designed based on the nucleotide sequence of the capture oligo. Although DNA is usually used as the nucleic acid probe, RNA may also be used. As the method for amplifying a nucleic acid, there can be mentioned, for example, a method of amplifying a nucleic acid as DNA by polymerase chain reaction (PCR) and a method of amplifying a nucleic acid as RNA by the in vitro transcription method.

The primers used in PCR are designed so that the nucleic acid probe should include a specific mutation site in the gyrA gene and sequences on both sides thereof. As such primers, there can be mentioned oligonucleotides having the nucleotide sequences shown as SEQ ID NOS: 5 and 6.

If a primer is labeled beforehand, a labeled nucleic acid probe can be obtained. The nucleic acid probe may also be labeled during or after the nucleic acid amplification reaction. As a labeling substance, labeling substances similar to those used for a probe employed in usual hybridization such as a fluorescence substance or hapten can be used. Specific examples of the fluorescence substance include fluorescein (FITC), Rhodamine, phycoerythrin (PE), Texas Red, cyanine fluorescent dyes and so forth. Specific examples of the hapten include biotin, digoxigenin (Dig), dinitrophenyl (DNP) and so forth.

The primers for preparing a nucleic acid probe can be included in a kit for determining quinolon resistance of tubercle bacilli together with a substrate on which the oligonucleotide is immobilized.

The hybridization can be performed in the same manner as in usual nucleic acid hybridization. A specific method will be exemplified below.

A nucleic acid probe is added to a mixed solution containing a salt solution such as SSC (standard saline citrate), a blocking solution containing sodium dodecylsulfate (SDS), bovine serum albumin (BSA) or the like and additives for promoting a hybridization reaction. When the target is a double strand, denaturation is performed by heating or the like. A nucleic acid probe solution is added onto a substrate in an amount of several μL and heated for several hours (usually 37-70° C.) to form a hybrid between an oligonucleotide immobilized on the substrate and the nucleic acid probe.

A solution of 5×SSC or 3 M tetramethylammonium chloride is added onto the substrate and heated (usually 37-50° C.) to remove oligonucleotides that form a nonspecific hybrid or do not form specific hybrid from the substrate so that only specific hybrids should be selectively left on the substrate.

The hybrids are detected by using the fluorescence substance or hapten introduced into the nucleic acid probe. When a hapten is used, a solution containing a conjugate of a protein that recognizes the hapten or protein that bonds to the hapten and alkaline phosphatase, horseradish peroxidase or the like (enzyme conjugate) is added onto the substrate and allowed to react at room temperature for several dozens of minutes. A nonspecific adsorption reaction of the enzyme conjugate and the substrate can be prevented by completely coating regions on the substrate with a protein such as BSA except for the regions in which the oligonucleotides are immobilized before a bonding reaction of the hapten and the enzyme conjugate is performed. This treatment can be performed by, after the oligonucleotides are immobilized, adding a solution of a protein such as BSA onto the substrate and leaving it at room temperature for several dozens of minutes. After the bonding reaction of the enzyme conjugate and the hapten of the nucleic acid probe is completed, only enzyme conjugates that bond to the hapten in the nucleic acid probe are left on the substrate by washing the substrate with an appropriate buffer containing a surfactant to remove enzyme conjugates that did not bond to the hapten.

In order to visualize the hybrids, a compound that becomes insoluble only when only a bonding product of the hapten and the enzyme conjugate exists is added. The production of the insoluble compound is amplified by the enzymatic reaction, and thus the hybrids are visualized. As the compound used for this purpose, nitroblue tetrazolium chloride (NBT) and 5-bromo-4-chloro-3-indolyl phosphate, p-toluidine salt (BCIP) are used when the enzyme in the enzyme conjugate is alkaline phosphatase. When the enzyme is horseradish peroxidase, 3,3′,5,5′-tetramethylbenzidine (TMB) or the like can be used.

Quinolon resistance of tubercle bacillius is determined based on the obtained results of the hybridization by examining pigmentation or fluorescent color development at positions at which the capture oligos are immobilized.

The kit of the present invention is a kit for determining quinolon resistance of a tubercle bacillus strain by detecting a mutation in the gyrA gene of the bacterial strain, which includes an oligonucleotide for detecting a substitution of another amino acid residue for an amino acid residue corresponding to the 89th aspartic acid residue in the amino acid sequence encoded by the gyrase A gene. As the oligonucleotide, a capture oligo or a substrate on which the capture oligo is immobilized can be mentioned. Further, the kit of the present invention may include primers for preparing a nucleic acid probe, labeled nucleic acid probe, buffer, reagents for hybridization such as enzyme conjugate that recognizes hapten and so forth.

As quinolon to which the present invention can be applied, there can be mentioned levofloxacin, ciprofloxacin, ofloxacin, gatifloxacin and so forth.

### EXAMPLES

Hereafter, the present invention will be explained more specifically with reference to the following example.

<1> Isolation of Quinolon-resistant Strain of Mycobacterium tuberculosis

Sputum of a tuberculosis patient was applied on Ogawa medium and cultured in an incubator at 37° C. for 4 weeks. Appeared colonies were inoculated again on Ogawa medium containing 2.5 μg/mL of levofloxacin and cultured in an incubator at 37° C. for 4 weeks. The strains that formed colonies were isolated as quinolon-resistant strains.

<2>Determination of Nucleotide Sequence of gyrA Gene of Quinolon-resistant Strain

Genomic DNA was prepared from each of the quinolon-resistant strains isolated as described above by the method of Kent et al. (Kent, P. T., et al., Mycobacteriology, A Guide for the Level III Laboratory, p.31, U.S. Department of Health and Human Service, Public Health Service, Center for Disease Control, 1985). PCR was performed for the obtained genome by using primers having the nucleotide sequences shown as SEQ ID NOS: 5 and 6.

rTaq (Takara Shuzo) was used for PCR. PCR was performed by a reaction at 98° C. for 1 minute, 35 cycles of reactions at 98° C. for 5 seconds, at 55° C. for 10 seconds and at 72° C. for 20 seconds, and a reaction at 72° C. for 1 minute. The amplification product was separated by 1% agarose gel electrophoresis, and a gel containing a 0.4 kbp band was excised. This gel piece was placed into a 1.5-mL micro tube and frozen at −135° C. for 15 minutes. Then, the gel was centrifuged at 15000 rpm for 10 minutes to obtain a supernatant. Resulting DNA solution was used as a template together with a primer shown as SEQ ID NO: 5 and Big Dye Terminator kit manufactured by ABI to perform a sequencing reaction. After completion of the reaction, the sequence was analyzed by using a genetic analyzer manufactured by ABI.

FIG. 1 shows alignment of the obtained sequences. In FIG. 1, the number shown at the right end of each sequence represents the number of sequences that had the same mutation among the determined sequences. Among these mutations, substitution of A for G at the 530th position in the nucleotide sequence shown as SEQ ID NO: 3 is a novel mutation. Further, substitution of A for G at the 530th position and substitution of A for G at the 545th position constitute a novel combination of mutations. Other mutations were the same as the mutations already reported (Antimicrob. Agents Chemother., 1996, 40 (8), 1768-1774; J. Infect. Dis., 1996, 174, 1127-1130; Eur. J. Clin. Microbiol. Infect. Dis., 1997, 16, 395-398; J. Infect. Dis., 2000, 182, 517-525).

What is claimed is:

1. A method for determining quinolon resistance of a Mycobacterium tuberculosis strain comprising detecting a mutation in a gyrase A gene of the bacterial strain, wherein the mutation is substitution of another amino acid residue for an amino acid residue corresponding to the 89th aspartic acid residue in the amino acid sequence encoded by the gyrase A gene.

2. The method according to claim 1, wherein the other amino acid residue is an asparagine residue.

3. The method according to claim 1 or 2, wherein the amino acid sequence encoded by the gyrase A gene is the amino acid sequence of SEQ ID NO: 4 or an amino acid sequence of SEQ ID NO: 4 including substitution, deletion or insertion of one or several amino acid residues.

4. The method according to any one of claims 1 to 3, wherein the mutation is substitution of T for G that is the first nucleotide of a codon coding for an aspartic acid residue.

5. The method according to claim 4, wherein the mutation occurs at a position corresponding to the 530th nucleotide in the nucleotide sequence of SEQ ID NO: 3.

6. The method according to any one of claims 1 to 5, further comprising detecting a substitution of A for a base corresponding to G at the 545th position in the nucleotide sequence of SEQ ID NO: 3.

7. A kit for determining quinolon resistance of a Mycobacterium tuberculosis strain by detecting a mutation in a gyrase A gene of the bacterial strain, comprising an oligonucleotide for detecting a substitution of another amino acid residue for an amino acid residue corresponding to the 89th aspartic acid residue in an amino acid sequence encoded by the gyrase A gene.


SEQUENCE LISTING (6 sequences)

SEQ ID NO: 1 - DNA, 50 bp, Mycobacterium tuberculosis
  CDS (2)..(49)
caactaccacccgcacggcgacgcgtcgatctacgacagcctggtgcgca

SEQ ID NO: 2 - protein, 16 aa, Mycobacterium tuberculosis
NYHPHGDASIYDSLVR

SEQ ID NO: 3 - DNA, 2884 bp, Mycobacterium tuberculosis
  CDS (266)..(2782)
aagatcaacaaggaagacggcattcagcggtacaagggtctaggtgaaatggacgctaaggagttgtgggagaccaccatggatccctcggttcgtgtgttgcgtcaagtgacgctggacgacgccgccgccgccgacgagttgttctccatcctgatgggcgaggacgtcgacgcgcggcgcagctttatcacccgcaacgccaaggatgttcggttcctggatgtctaacgcaaccctgcgttcgattgcaaacgaggaatagatgacagacacgacgttgccgcctgacgactcgctcgaccggatcgaaccggttgacatcgagcaggagatgcagcgcagctacatcgactatgcgatgagcgtgatcgtcggccgcgcgctgccggaggtgcgcgacgggctcaagcccgtgcatcgccgggtgctctatgcaatgttcgattccggcttccgcccggaccgcagccacgccaagtcggcccggtcggttgccgagaccatgggcaactaccacccgcacggcgacgcgtcgatctacgacagcctggtgcgcatggcccagccctggtcgctgcgctacccgctggtggacggccagggcaacttcggctcgccaggcaatgacccaccggcggcgatgaggtacaccgaagcccggctgaccccgttggcgatggagatgctgagggaaatcgacgaggagacagtcgatttcatccctaactacgacggccgggtgcaagagccgacggtgctacccagccggttccccaacctgctggccaacgggtcaggcggcatcgcggtcggcatggcaaccaatatcccgccgcacaacctgcgtgagctggccgacgcggtgttctgggcgctggagaatcacgacgccgacgaagaggagaccctggccgcggtcatggggcgggttaaaggcccggacttcccgaccgccggactgatcgtcggatcccagggcaccgctgatgcctacaaaactggccgcggctccattcgaatgcgcggagttgttgaggtagaagaggattcccgcggtcgtacctcgctggtgatcaccgagttgccgtatcaggtcaaccacgacaacttcatcacttcgatcgccgaacaggtccgagacggcaagctggccggcatttccaacattgaggaccagtctagcgatcgggtcggtttacgcatcgtcatcgagatcaagcgcgatgcggtggccaaggtggtgatcaataacctttacaagcacacccagctgcagaccagctttggcgccaacatgctagcgatcgtcgacggggtgccgcgcacgctgcgactggaccagctgatccgctattacgttgaccaccaactcgacgtcattgtgcggcgcaccacctaccggctgcgcaaggcaaacgagcgagcccacattctgcgcggcctggttaaagcgctcgacgcgctggacgaggtcattgcactgatccgggcgtcggagaccgtcgatatcgcccgggccggactgatcgagctgctcgacatcgacgagatccaggcccaggcaatcctggacatgcagttgcggcgcctggccgcactggaacgccagcgcatcatcgacgacctggccaaaatcgaggccgagatcgccgatctggaagacatcctggcaaaacccgagcggcagcgtgggatcgtgcgcgacgaactcgccgaaatcgtggacaggcacggcgacgaccggcgtacccggatcatcgcggccgacggagacgtcagcgacgaggatttgatcgcccgcgaggacgtcgttgtcactatcaccgaaacgggatacgccaagcgcaccaagaccgatctgtatcgcagccagaaacgcggcggcaagggcgtgcagggtgcggggttgaagcaggacgacatcgtcgcgcacttcttcgtgtgctccacccacgatttgatcctgttcttcaccacccagggacgggtttatcgggccaaggcctacgacttgcccgaggcctcccggacggcgcgcgggcagcacgtggccaacctgttagccttccagcccgaggaacgcatcgcccaggtcatccagattcgcggctacaccgacgccccgtacctggtgctggccactcgcaacgggctggtgaaaaagtccaagctgaccgacttcgactccaatcgctcgggcggaatcgtggcggtcaacctgcgcgacaacgacgagctggtcggtgcggtgctgtgttcggccggcgacgacctgctgctggtctcggccaacgggcagtccatcaggttctcggcgaccgacgaggcgctgcggccaatgggtcgtgccacctcgggtgtgcagggcatgcggttcaatatcgacgaccggctcgtgtcgctgaacgtcgtgcgtgaaggcacctatctgctggtggcgacgtcagggggctatgcgaaacgtaccgcgatcgaggaatacccggtacagggccgcggcggtaaaggtgtgctgacggtcatgtacgaccgccggcgcggcaggttggttggggcgttgattgtcgacgacgacagcgagctgtatgccgtcacttccggcggtggcgtgatccgcaccgcggcacgccaggttcgcaaggcgggacggcagaccaagggtgttcggttgatgaatctgggcgagggcgacacactgttggccatcgcgcgcaacgccgaagaaagtggcgacgataatgccgtggacgccaacggcgcagaccagacgggcaattaatcaggctcgcccgacgacgatgcggatcgcgtagcgatctgaggaggaatcgggcagctaggctcggcagccgggtacgagtgttaggagtcggggtgactg

SEQ ID NO: 4 - protein, 838 aa, Mycobacterium tuberculosis
MTDTTLPPDDSLDRIEPVDIEQEMQRSYIDYAMSVIVGRALPEVRDGLKPVHRRVLYAMFDSGFRPDRSHAKSARSVAETMGNYHPHGDASIYDSLVRMAQPWSLRYPLVDGQGNFGSPGNDPPAAMRYTEARLTPLAMEMLREIDEETVDFIPNYDGRVQEPTVLPSRFPNLLANGSGGIAVGMATNIPPHNLRELADAVFWALENHDADEEETLAAVMGRVKGPDFPTAGLIVGSQGTADAYKTGRGSIRMRGVVEVEEDSRGRTSLVITELPYQVNHDNFITSIAEQVRDGKLAGISNIEDQSSDRVGLRIVIEIKRDAVAKVVINNLYKHTQLQTSFGANMLAIVDGVPRTLRLDQLIRYYVDHQLDVIVRRTTYRLRKANERAHILRGLVKALDALDEVIALIRASETVDIARAGLIELLDIDEIQAQAILDMQLRRLAALERQRIIDDLAKIEAEIADLEDILAKPERQRGIVRDELAEIVDRHGDDRRTRIIAADGDVSDEDLIAREDVVVTITETGYAKRTKTDLYRSQKRGGKGVQGAGLKQDDIVAHFFVCSTHDLILFFTTQGRVYRAKAYDLPEASRTARGQHVANLLAFQPEERIAQVIQIRGYTDAPYLVLATRNGLVKKSKLTDFDSNRSGGIVAVNLRDNDELVGAVLCSAGDDLLLVSANGQSIRFSATDEALRPMGRATSGVQGMRFNIDDRLVSLNVVREGTYLLVATSGGYAKRTAIEEYPVQGRGGKGVLTVMYDRRRGRLVGALIVDDDSELYAVTSGGGVIRTAARQVRKAGRQTKGVRLMNLGEGDTLLAIARNAEESGDDNAVDANGADQTGN

SEQ ID NO: 5 - DNA, 23 bp, Mycobacterium tuberculosis
  CDS (2)..(49)
agcgcagctacatcgactatgcg

SEQ ID NO: 6 - DNA, 23 bp, Mycobacterium tuberculosis
  CDS (2)..(49)
cttcggtgtacctcatcgccgcc